(19) United States
(12) Patent Application Publication
(10) Pub. No.: US 2015/0297502 A1
(43) Pub. Date: Oct. 22, 2015

(54) Cosmeceutical Formulations of Natural Ingredients for Hair Growth

(71) Applicant: AmbeRx Laboratories, Inc., San Diego, CA (US)

(72) Inventor: Yong Qiu, San Diego, CA (US)

(73) Assignee: AMBERX LABORATORIES, INC., San Diego, CA (US)

(21) Appl. No.: 14/258,264

(22) Filed: Apr. 22, 2014

(51) Int. Cl. A61K 8/97; A61Q 7/00; A61K 8/35

(52) U.S. Cl. CPC A61K 8/97; A61K 8/35; A61Q 7/00; A61K 2800/591

(57) ABSTRACT

The invention relates to a cosmeceutical composition comprising of natural ingredients which is useful as a topical formulation for improving the growth of human hair. The invention also provides a method for preparing the said composition as well as the suitable applications of the said composition.

### FIELD OF THE INVENTION

This invention relates generally to cosmeceutical compositions and methods for use in improving the growth of human hair. More particularly, the present invention provides compositions of the specific combination of natural ingredients and method of treatment for stimulating the human hair growth.

### BACKGROUND OF THE INVENTION

The cosmetic or pharmaceutical industry is always looking for compositions which can eliminate or reduce hair loss or stimulate hair growth. Hair loss is a problem suffered by many people because of a wide variety of reasons, naturally occurring, pathological induced or chemically promoted.

Many studies and researches have been done to understand the hair loss problem. It is now believed that hair loss, or generally known as alopecia is a result of combining factors associated with disruption of hair growing cycles, transition of hair types, and diminution of hair follicles.

To understand the cause for hair loss, it is necessary to first recognize the process of hair growth. Human hair growth is characterized as a cycle of different phases with different activity of growth and rest. Unlike many mammals, human hair growth is asynchronous in the growing cycle, such that some hair follicles are growing while others are dormant. The growing cycle can be divided into three main stages; anagen phase, catagen phase, and telogen phase. Anagen phase is the growth phase of the cycle and is characterized by penetration of the hair follicle deep into the dermis with rapid proliferation of cells which are differentiating to form hair. The duration of the anagen phase is not the same for human hairs at different sites. For the case of scalp hair, the anagen phase generally lasts for 6-8 years, whereas for the case of modified terminal hair like eyelash and eye brows, it only lasts about 5-12 months. The next is the catagen phase, which is a transitional stage marked by the cessation of cell division, and during which the hair follicle regresses through the dermis and hair growth ceases. The final phase is the telogen phase, characterized as the resting stage during which all hair growth ceases and the hair eventually is shed preparatory to the follicle commencing to grow a new one. Natural dropping or loss of hair can be estimated at about one hundred hairs, on average, per day for a normal physiological condition. However, a wide variety of conditions can disrupt the normal ongoing physiological hair renewal process and cause substantial hair loss, temporary or permanent. Those conditions include androgenic alopecia, or common pattern baldness, anagen effluvium which is a chemotherapy-induced hair loss, telogen effluvium which is induced by stress, fever and drugs, and alopecia areata which is an autoimmune disease afflicting an estimated four million people.

To understand the hair loss problem, it is also necessary to recognize the various types of hair, including, terminal hairs and vellus hairs and modified terminal hairs, such as seen in eyelashes and eye brows. Terminal hairs are coarse, pigmented, long hairs in which the bulb of the hair follicle is seated deep in the dermis. Vellus hairs, on the other hand, are fine, thin, non-pigmented short hairs in which the hair bulb is located superficially in the dermis. Some of the hair loss problems are believed to be due to the transition of terminal hair to vellus hair.

Severe diminution of hair follicles is another main cause for hair loss. Comparing to a non-bald human subject who normally has an average of 460 follicles per square centimeter, a bald human subject in the same age group (30-90 years) only has about 306 follicles per square centimeter. This amounts to a one-third reduction in hair follicles which, when added to the increased number of hair follicles in telogen phase and increased proportion of vellus hair, is both significant and noticeable.

To treat the alopecia problem, various attempts have been made, including hormone adjustment, hair transplantation, ultraviolet radiation, massage, psychiatric treatment and exercise therapy. By far, the most common approach to the problem of hair loss, especially male pattern alopecia, has been drug therapy. Many types of drugs ranging from vitamins to hormones have been tried and some drug treatments have been demonstrated as showing promise in reversing male pattern alopecia. Currently, there are two drugs approved by the Food & Drug Administration (FDA) for the treatment of male pattern baldness: Rogaine (topical minoxidil) and Propecia (oral finasteride).

Minoxidil is a potassium channel agonist compound originally developed for treating severe hypertension because it potently induces peripheral vasodilation. U.S. Pat. No. 4,139,619 and U.S. Pat. No. 4,968,812 disclosed that minoxidil is also useful for the treatment of male pattern alopecia. Finasteride is a 5-α reductase inhibitor compound originally used to treat urinary problems caused by benign prostatic hyperplasia. U.S. Pat. No. 5,547,957 and U.S. Pat. No. 5,571,817 disclosed the methods of treating androgenic alopecia using finasteride. Finasteride can block the activity of 5-α reductase, an enzyme that converts testosterone to dihydrotestosterone (DHT) which has been implicated in miniaturization of hairs, a precursor to catagen. Minoxidil and finasteride both stimulate hair growth in some patients, but only for the duration of drug use. New hair growth ends and hair loss resumes shortly after the patient stops treatment. After several months' use, minoxidil successfully induces limited hair growth for approximately 1 in 3 patients, and slows hair loss for roughly 9 in 10. Oral finasteride is generally more effective than topical minoxidil at inducing hair growth, but both treatments are far less than 100% effective. Further hair loss is prevented in most patients treated with finasteride. About half of treated patients achieve some hair growth, and approximately one-third of patients experience cosmetically improved hair growth after two years of continuous use.

Furthermore, there are other patents describing the use of topical vasodilators intended to stimulate hair growth by improving the microcirculation of the scalp. These patents include using NO (nitrogen monoxide) donors for applying to the scalp (EP0 327 263), or using arginine and its derivatives as an NO-synthase substrate for the in vivo formation of NO which in turn improving the microcirculation (WO 99/13717; JP 07316023) on the scalp for the treatment of alopecia.

There are also patents describing the use of prostaglandins and their derivatives or analogs for treating hair loss or enhancing hair growth. Prostaglandins in general have a wide range of biological activities. For example, PGE₂ has such properties as regulators of cell proliferation, regulators of cytokine synthesis, regulators of immune responses, and inducers of vasodilatation. Prostaglandin derivatives have also been disclosed having vasodilation properties in U.S. Pat. No. 4,311,707, U.S. Pat. No. 5,288,754 and U.S. Pat. No. 5,532,708. Prostaglandins and derivatives thereof have been suggested useful in enhancing hair growth as provided in U.S. Pat. No. 6,262,105. Prostaglandin analogs or non-naturally occurring prostaglandins have been suggested useful for stimulating eyelash growth (U.S. Pat. No. 7,351,404, U.S. Pat. No. 7,388,029, and U.S. Pat. No. 8,038,988) or enhancing hair growth by conversion of vellus hair or intermediate hair to growth as terminal hair (U.S. Pat. No. 8,101,161) or reversing hair loss (U.S. Pat. No. 8,541,466).

Like all drug therapies, using synthetic chemical compound treating hair loss or enhancing hair growth can be associated with undesired side effects even if the methods are shown to be effective. For example, taking finasteride orally to treat alopecia can result in decreased sexual ability or desire. In the case of using prostaglandin analogs or non-naturally occurring prostaglandins such as a prostaglandin F analog or to be specifically a cyclopentane heptanoic acid, 2-cycloalkyl or aryl alkyl compound as suggested in U.S. Pat. No. 8,101,161 to treat eyelash growth can lead to eye irritation and skin darkening. Even in the case of using naturally occurring prostaglandins can cause side effects such as inflammation, surface irritation, smooth muscle contraction, pain, and bronchoconstriction. Because of these limitations, there is a significant need for new compositions and methods for promoting hair growth which are non-toxic and free of undesirable side effects.

### SUMMARY OF THE INVENTION

The invention provides a composition for use as a topical formulation to stimulate human hair growth.

The invention also provides a composition comprising a specific combination of natural ingredients of natural plant extracts with high level of gamma-linolenic acid (GLA) and a biodegradable coating agent to encapsulate the natural ingredients.

The invention also provides that the natural ingredients are selected from borage seed oil, evening primrose oil, blackcurrant seed oil, hemp seed oil, GLA safflower oil and a combination thereof. The natural ingredients is about 0.001% to 20% by weight of the total formulation; preferably 0.01% to 10% by weight of the total formulation.

The invention also provides that the natural ingredients are preferably selected from borage seed oil, evening primrose oil and blackcurrant seed oil and a combination thereof. The combinations of the three natural ingredients have a weight ratio in the range from 0.01:1:0.01 to 100:1:100; preferably in the range from 0.05:1:0.05 to 20:1:20; and most preferably in the range from 0.1:1:0.1 to 10:1:10.

The invention also provides that the biodegradable coating agent is a copolymer selected from the group consisting of lactide, glycolide and caprolactone. The biodegradable coating agent is about 0.01% to 10% by weight of the total formulation.

The invention further provides an optional ingredient, curcumin. The curcumin is in the range of 0.01% to 5% by weight of the total formulation.

The invention also provides a method of stimulating human hair growth by administering topically an effective amount of a composition of the invention. The compositions can be applied to skin or scalp; the human hair can be eyelash, eyebrow and scalp hair.

The invention also provides a method for preparing the compositions of the invention, including the steps of:
(a) preparing an oil in water micro-emulsion with the natural ingredients in the oil phase;
(b) preparing a polymer solution with a water-insoluble biodegradable polymer dissolved in a water-miscible solvent or co-solvents;
(c) preparing an aqueous solution containing emulsion stabilizers;
(d) while agitating, adding the emulsion obtained in step (a) into the polymer solution obtained in step (b), and adding the emulsion stabilizer into the aqueous solution obtained in step (c); and
(e) rinsing with an excess amount of water to wash off the organic solvent, and diluting the resulted emulsion with purified water to pre-determined concentration of the natural ingredients.

In one aspect, the water-miscible solvent or co-solvents are selected from acetic acid, ethyl acetate, glycofurol, N-methylpyrrolidone, triacetin, and benzyl alcohol. In another aspect, the emulsion stabilizer is selected from vitamin E TPGS, polyvinyl alcohol (PVA), sodium lauryl sulfate, lauryl phosphate, ethoxylated sorbates such as Tween-80, polyglycerol and polyethylene glycol, or a mixture thereof.

### DETAILED DESCRIPTION OF THE INVENTION

The present invention provides stable cosmeceutical compositions which can be used for topical application to stimulate human hair growth. The present invention also provides a novel and effective method for promoting hair growth using the composition of a natural ingredient combination as topical application onto the skin or scalp. The present invention employs micro-emulsion solvent removal method to prepare nanocapsules, in which natural ingredients are surrounded and coated by a biodegradable polymer membrane.

The composition according to the present invention contains natural ingredients which are preferably selected from natural plant extracts with high level gamma-linolenic acid (GLA). Such plant extracts include natural oil ingredients such as borage seed oil, evening primrose oil, blackcurrant seed oil, hemp seed oil and GLA safflower oil. The composition according to the present invention preferably contains an optimized combination of the aforementioned natural ingredients. The total natural ingredients may represent from 0.001% to 20% by weight, preferably from 0.01% to 10% by weight on the total final weight of the composition. The natural ingredients are preferably selected from borage seed oil, evening primrose oil and blackcurrant seed oil and a combination thereof. Their respective weight ratio in the combination are in the range from 0.01:1:0.01 to 100:1:100 and more preferably from 0.05:1:0.05 to 20:1:20, and most preferably from 0.1:1:0.1 to 10:1:10. Furthermore, an additional natural ingredient may optionally be added to the composition to enhance the effectiveness of human hair growth. The additional natural ingredient is preferably curcumin.

The biodegradable polymer of the present invention can serve as the membrane forming material. The biodegradable polymer is preferably selected from poly-lactides, poly-glycolides, poly-lactide-co-glycolides-, polycaprolactone, polycarbonates, polyesteramides, poly-amino acids, polyorthoesters, polycyanoacrylates, biodegradable polyurethanes, blends and copolymers thereof. The most preferred polymers are poly-lactides, poly-glycolides, copolymers of lactides and glycolides, terpolymers of lactides and glycolides, blends thereof, or mixtures thereof.

The method for preparing the composition in a stable nanocapsule form according to the present invention comprises steps of:
(i) preparing an oil in water emulsion with the natural ingredients in the oil phase;
(ii) preparing a polymer solution with a water-insoluble polymer dissolved in a water-miscible solvent or co-solvents comprised of a mixture of the water-miscible solvents, which is selected from acetic acid, ethyl acetate, glycofurol, N-methylpyrrolidone, triacetin, and benzyl alcohol.
(iii) preparing an aqueous solution saturated with the same organic solvent used in step (ii), containing emulsion stabilizers;
(iv) while under agitation, adding the emulsion obtained in step (i) into the polymer solution obtained in step (ii), in order to deposit the polymer on the interface of the emulsion to encapsulate the natural oil ingredients, and then immediately adding the resulted emulsion into the aqueous solution obtained in step (iii);
(v) rinsing the resulted emulsion obtained from step (iv) with excessive amount of purified water to eliminate the acetic acid and ethyl acetate, and then diluting the emulsion with purified water to pre-determined concentration of the natural ingredients.

In step (iii), the emulsion stabilizer refers to a compound that prevents the separation of the emulsion into individual oil and aqueous phases. The emulsion stabilizer is selected from vitamin E TPGS, polyvinyl alcohol (PVA), sodium lauryl sulfate, lauryl phosphate, ethoxylated sorbates such as Tween-80, polyglycerol and polyethylene glycol, and/or a mixture thereof. It may be necessary to adjust the pH of the aqueous solution of the final composition for topical administration.

The nanocapsules of the present invention are effective to protect the natural ingredient combination and to provide sufficient stability until being administered onto the skin. A typical nanocapsule has a size dimension less than 1 micron in size, more generally in the range of about 0.05 micron to about 0.9 micron.

The present invention also relates to a method of using the aforementioned composition of natural ingredients as topical formulation for promoting hair growth. The term “topical” as employed herein relates to the use of a composition incorporated in a suitable carrier, and applied at the site of thinning hair or baldness for exertion of local action. Accordingly, such topical compositions include those natural ingredients in which the natural ingredients are applied externally by direct contact with the skin surface to be treated. Conventional cosmetic formulations for this purpose include ointments, liniments, creams, shampoos, lotions, pastes, jellies, sprays, aerosols, and the like depending on the part of the body to be treated. Typically, the composition of the present invention needs to be applied repeatedly for a sustained period of time on the part of the body to be treated, for example, the eyelids, eyebrows, skin or scalp. The preferred method will generally involve regular, such as daily, administration for a period of at least two weeks, more preferably at least four weeks, and most preferably at least eight weeks.

For topical use on the eyelids or eyebrows, the composition of the present invention can be further diluted or formulated in aqueous solutions, creams, ointments or oils. Such formulations may or may not, depending on the dispenser, further contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. Furthermore, aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol.

The foregoing will be better understood with reference to the following examples which detail certain procedures for the manufacture of compositions according to the present invention. All references made to these examples are for the purposes of illustration. They are not to be considered limiting as to the scope and nature of the present invention.

### EXAMPLES

### Example 1

TABLE 1
Compositions of Formulation 1

| Component | % (w/w) |
|---|---|
| Borage seed oil | 0.20% |
| Evening primrose oil | 0.10% |
| Blackcurrant seed oil | 0.20% |
| Egg lecithin (LIPOID E80) | 0.50% |
| Vitamin E TPGS (a) | 1.05% |
| Poly(lactic acid-co-glycolic acid) (50:50) (PLGA) | 0.90% |
| Vitamin E TPGS (b) | 0.71% |
| Purified water | Add to 100% |

A micro-emulsion was prepared, according to Table 1 listed percentage, by dissolving 0.2 g of borage seed oil, 0.1 g of evening primrose oil, 0.2 g blackcurrant seed oil, 0.5 g egg lecithin in appropriate amount of ethyl acetate to form an oil phase. An aqueous phase was prepared by dissolving 1.05 g vitamin E TPGS (a) in appropriate amount of water. The aqueous phase was added to the oil phase, and the mixture was agitated using a homogenizer to obtain a crude emulsion. The crude emulsion was passed 3 times through a microfluidizer equipped with an emulsion interaction chamber to form a micro-emulsion. The micro-emulsion formed in this process was a translucent liquid.

The biodegradable polymer solution was prepared by dissolving 0.9 g of poly-lactic acid-co-glycolic acid (50:50) (PLGA) according to Table 1 into ethyl acetate: acetic acid (30:70) co-solvent solution.

The second aqueous phase was prepared by dissolving 0.71 g Vitamin E TPGS (b) into appropriate amount of purified water containing ethyl acetate.

While under agitating, the micro-emulsion was added drop wise to the polymer solution. Upon completion of adding the micro-emulsion, the resulted mixture emulsion was immediately added to the aqueous phase. Because acetic acid is highly miscible with water, it diffused into the aqueous phase of the oil-in-water micro-emulsion. At the same time, water diffused out of the micro-emulsion to the polymer/co-solvent solution. With adding the resulted mixture into abundant aqueous phase, a further solvent diffusion process led to PLGA polymer redistributing to the surface of the micro-emulsion oil-water interface and precipitating due to the loss of the solvent. In the consequence, during this solvent diffusion process, PLGA polymer formed a coating membrane surrounding the natural ingredients oils by encapsulating them.

Purified water was then added to dilute the emulsion to predetermined concentration of the natural ingredients.

### Example 2

TABLE 2.1
Compositions of Formulation 2

| Component | % (w/w) |
|---|---|
| Borage seed oil | 0.15% |
| Evening primrose oil | 0.15% |
| Blackcurrant seed oil | 0.15% |
| Soy lecithin (Phospholipon 90 G) | 0.45% |
| Vitamin E TPGS (a) | 0.95% |
| Poly(lactic acid-co-glycolic acid) (50:50) PLGA) | 0.90% |
| Vitamin E TPGS (b) | 0.71% |
| Purified water | Add to 100% |

TABLE 2.2
Compositions of Formulation 3

| Component | % (w/w) |
|---|---|
| Borage seed oil | 0.15% |
| Evening primrose oil | 0.30% |
| Blackcurrant seed oil | 0.10% |
| Egg lecithin (LIPOID E80) | 0.55% |
| Polyvinyl alcohol (PVA) (a) | 1.16% |
| Poly(lactic acid-co-glycolic acid) (50:50) (PLGA) | 0.95% |
| Polyvinyl alcohol (PVA) (b) | 0.71% |
| Purified water | Add to 100% |

Two other formulations were prepared using the similar method of preparation as described in Example 1.

### Example 3

The formulation according to composition ratio in Table 2.1 was further diluted with a topical solution using the composition ratio of Table 3.

TABLE 3
Compositions of a Topical Formulation

| Component | % (w/w) |
|---|---|
| Natural ingredients composition | 0.346% |
| from Example 2 (as aqueous solution) | |
| Sodium phosphate dibasic anhydrous | 0.350% |
| Citric acid anhydrous | 0.010% |
| Sodium chloride | 0.800% |
| Benzalkonium Chloride | 0.010% |
| Purified water | Add to 100% |

A topical solution was prepared by weighing 900 mL of purified water in a suitable container. 10 mL of the composition prepared from Formulation 2 was sequentially added, with 0.35 g sodium phosphate dibasic anhydrous, 1 mL of 0.01% (w/w) citric acid solution (prepared by dissolving 1 g of citric acid into 100 mL purified water), 0.8 g sodium chloride and 0.01 g benzalkonium chloride. The pH of the resulted solution was adjusted to 7.0 to 7.2, and purified water was added to a final volume of 100 mL. The resulted topical solution can be directly administered on human skin to stimulate hair growth, such as eyelash growth or eyebrow growth.

TABLE 4
Compositions of Formulation 5

| Component | % (w/w) |
|---|---|
| Borage seed oil | 0.20% |
| Evening primrose oil | 0.10% |
| Blackcunant seed oil | 0.20% |
| Curcumin | 0.30% |
| Egg lecithin (LIPOID E80) | 0.50% |
| Vitamin E TPGS (a) | 1.15% |
| Poly(lactic acid-co-glycolic acid) | 0.93% |
| (50:50) (PLGA) | |
| Vitamin E TPGS (b) | 0.72% |
| Purified water | Add to 100% |

Composition of Formulation 5 was prepared using the similar method as described in Example 1, with 0.30 g of curcumin added in the oil phase.

### Example 5

Formulation 5 was prepared as a topical cream using the composition ratio of Table 5.

TABLE 5
Composition of a Cream Formulation

| Component | % (w/w) |
|---|---|
| Natural ingredients composition from | 0.41% |
| Formulation 5 (as aqueous solution) | |
| Propylene Glycol | 35.20% |
| Sodium Lauryl sulfate | 0.90% |
| Cetyl Alcohol | 1.00% |
| Germaben II | 0.30% |
| Purified water | 62.19% |

A topical cream was prepared by dissolving 0.9 g sodium lauryl sulfate into 52.6 mL of purified water in a suitable container; propylene glycol was added while mixing. The solution was heated to 65° C. Cetyl alcohol was melted in a separate container and maintained the temperature at 60° C. While agitating, the aqueous solution was added to the melted cetyl alcohol solution, and Germaben II was added into the resulted mixture and continued mixing during the cooling process. When the temperature reached 20° C., 10 mL of Formulation 5 was added and mixed well. The resulted topical cream can be directly administered on human scalp to stimulate hair growth.

### Example 6

### Topical Treatment of Volunteers

A study was done to evaluate the appearance of eyelashes around the eyes of healthy volunteers. A total of 5 volunteers, all female, average age 34 years, (ranging from 28-38 years) were recruited in the study. Each volunteer applied the topical solution of Formulation 5 onto the margin of their upper eyelids in the morning and evening daily for a total period of 28 days.

The length of the eyelashes of each volunteer was recorded before and after and treatment. After 28 days treatment, increased length of eyelashes was observed. The increase of eyelash length was ranging from 1 mm to 1.7 mm, representing 12% to 21% increase. Increased numbers of hairs, and more thickly appearance were also observed after the treatment.

Although specific embodiments of the invention have been described herein for purposes of illustration, various modifications may be made without deviating from the spirit and scope of the invention. Accordingly, the invention is not limited by the appended claims.

What is claimed is:

1. A composition for use as a topical formulation to stimulate human hair growth, the composition comprising:
(a) a specific combination of natural ingredients of natural plant extracts with high level of gamma-linolenic acid (GLA); and
(b) a biodegradable coating agent to encapsulate the natural ingredients.

2. The composition according to claim 1, wherein the natural ingredients are selected from the group consisting of borage seed oil, evening primrose oil, blackcurrant seed oil, hemp seed oil, GLA safflower oil and a combination thereof.

3. The composition according to claim 2, wherein the natural ingredients are preferably selected from the group consisting of borage seed oil, evening primrose oil and blackcurrant seed oil and a combination thereof.

4. The composition according to claim 1, wherein the natural ingredients is about 0.001% to 20% by weight of the total formulation.

5. The composition according to claim 3, wherein the combination of natural ingredients having a weight ratio in the range from 0.01:1:0.01 to 100:1:100.

6. The composition according to claim 5, wherein the combination of natural ingredients preferably having a weight ratio in the range from 0.05:1:0.05 to 20:1:20.

7. The composition according to claim 6, wherein the combination of natural ingredients preferably having a weight ratio in the range from 0.1:1:0.1 to 10:1:10.

8. The composition according to claim 1, wherein the biodegradable coating agent is a copolymer.

9. The composition according to claim 8, wherein the copolymer is selected from the group consisting of lactide, glycolide and caprolactone.

10. The composition according to claim 1, wherein the biodegradable coating agent is about 0.01% to 10% by weight of the total formulation.

11. The composition according to claim 1, further comprising curcumin.

12. The composition according to claim 11, wherein curcumin is in the range of 0.01% to 5% by weight of the total formulation.

13. A method of stimulating human hair growth comprising administering topically an effective amount of the composition of claim 1.

14. The method of claim 13, wherein the administering topically is on skin and scalp.

15. The method of claim 13, wherein the human hair is eyelash, eyebrow and scalp hair.

16. A method for preparing the composition of claim 1, comprising the steps of:
(a) preparing an oil in water micro-emulsion with the natural ingredients in the oil phase;
(b) preparing a polymer solution with a water-insoluble biodegradable polymer dissolved in a water-miscible solvent or co-solvents;
(c) preparing an aqueous solution containing emulsion stabilizers;
(d) while agitating, adding the emulsion obtained in step (a) into the polymer solution obtained in step (b), and adding the emulsion stabilizer into the aqueous solution obtained in step (c); and
(e) rinsing with an excess amount of water to wash off the organic solvent, and diluting the resulted emulsion with purified water to pre-determined concentration of the natural ingredients.

17. The method according to claim 16, wherein the water-miscible solvent or co-solvents are selected from acetic acid, ethyl acetate, glycofurol, N-methylpyrrolidone, triacetin, and benzyl alcohol.

18. The method according to claim 16, wherein the emulsion stabilizer is selected from vitamin E TPGS, polyvinyl alcohol (PVA), sodium lauryl sulfate, lauryl phosphate, ethoxylated sorbates such as Tween-80, polyglycerol and polyethylene glycol, and a mixture thereof.
